(19) United States
(12) Patent Application Publication
(10) Pub. No.: US 2022/0118615 A1
(43) Pub. Date: Apr. 21, 2022

(54) CONTROL APPARATUS, CONTROL METHOD, AND MASTER-SLAVE SYSTEM

(71) Applicant: Sony Group Corporation, Tokyo (JP)

(72) Inventor: Shunsuke YAJIMA, Tokyo (JP)

(73) Assignee: Sony Group Corporation, Tokyo (JP)

(21) Appl. No.: 17/431,714

(22) PCT Filed: Feb. 17, 2020
(86) PCT No.: PCT/JP2020/005970

§ 371 Date: Aug. 18, 2021

(30) Foreign Application Priority Data
Mar. 28, 2019 (JP) 2019-065060

(51) Int. Cl. B25J 9/16; B25J 9/10; B25J 3/04; A61B 34/37

(52) U.S. Cl. CPC B25J 9/1633; B25J 9/104; B25J 3/04; A61B 2034/302; B25J 9/1653; A61B 34/37; B25J 9/1605

(57) ABSTRACT

Provided is a control apparatus configured to control a parallel wire mechanism.

The control apparatus for a parallel wire apparatus configured to pull a movable portion with a plurality of wires decomposes a control model in which the movable portion is driven by a pair of opposed motors with use of the wires to a center of gravity mode in which a motor C is controlled to make the movable portion achieve desired acceleration and a relative mode in which a motor R is controlled to make an elastic force that acts on the wires constant, by mode decomposition, and performs coordinate transformation on an acceleration reference value for the motor C determined in the center of gravity mode and an acceleration reference value for the motor R determined in the relative mode, to thereby obtain an acceleration reference value for the pair of motors.

### TECHNICAL FIELD

The technology disclosed herein relates to a control apparatus and a control method for controlling a parallel wire mechanism and to a master-slave system including a master apparatus and a slave apparatus at least one of which includes the parallel wire mechanism.

### BACKGROUND ART

As small inertia drive systems, the parallel wire system and the parallel link system have been known. These parallel mechanisms can be used in, for example, a master-slave system to drive a controller that the operator operates on the master side and a device at the output end on the slave side (end effector). The parallel wire system is generally smaller in inertia than the parallel link system.

Further, as a control system for master-slave systems, the bilateral system in which the slave apparatus is operated from the master apparatus while the state of the slave apparatus is fed back to the master apparatus (for example, see PTL 1) has been known.

To achieve the bilateral control system, highly accurate simultaneous control of position and force is required. However, in the case of parallel wire mechanisms, there is a concern of deterioration of the control accuracy due to vibration or elongation unique to the wires. For example, there has been proposed a technology for achieving, in a parallel wire driven robot, position control and force control individually while preventing vibration (for example, see NPL 1). The position control and the force control, however, are not performed simultaneously.

### CITATION LIST

### Patent Literature

[PTL 1]
Japanese Patent Laid-open No. 2019-034002

### Non Patent Literature

[NPL 1]
Hitoshi Kino and others, “A Motion Control Scheme in Task Oriented Coordinates and its Robustness for Parallel Wire Driven Systems” (Journal of the Robotics Society of Japan, Vol. 18, No. 3, pp. 411-418, 2000)

### SUMMARY

### Technical Problem

It is an object of the technology disclosed herein to provide a control apparatus and a control method for controlling a parallel wire mechanism while preventing vibration and elongation unique to the wires as well as a master-slave system that includes a master apparatus and a slave apparatus at least one of which includes the parallel wire mechanism and that performs bilateral control.

### Solution to Problem

According to a first aspect of the technology disclosed herein, there is provided a control apparatus for a parallel wire apparatus configured to pull a movable portion with a plurality of wires, the control apparatus being configured to control force and a position of the movable portion, based on acceleration.

The control apparatus according to the first aspect decomposes a control model in which the movable portion is driven by a pair of opposed motors with use of the wires to a center of gravity mode in which a motor C is controlled to make the movable portion achieve desired acceleration and a relative mode in which a motor R is controlled to make an elastic force that acts on the wires constant, by mode decomposition, and performs coordinate transformation on an acceleration reference value for the motor C determined in the center of gravity mode and an acceleration reference value for the motor R determined in the relative mode, to thereby obtain an acceleration reference value for the pair of motors.

Further, according to a second aspect of the technology disclosed herein, there is provided a control method for a parallel wire apparatus configured to pull a movable portion with a plurality of wires, the control method including steps of controlling, by a control system, a motor C in a center of gravity mode to make the movable portion achieve desired acceleration, the control system being configured to independently control an acceleration response and tension of the wires in a control model in which the movable portion is driven by a pair of opposed motors with use of the wires;

controlling, by the control system, a motor R in a relative mode to make an elastic force that acts on the wires constant; and performing, by the control system, acceleration control on the pair of motors, based on acceleration reference values for the motor C and the motor R.

Further, according to a third aspect of the technology disclosed herein, there is provided a master-slave system including a master apparatus and a slave apparatus at least one of which includes a parallel wire mechanism configured to pull a movable portion with a plurality of wires; and a control apparatus configured to control force and a position of the movable portion, based on acceleration, while preventing elongation and vibration of the wires. The control apparatus constitutes a control system configured to independently control an acceleration response and tension of the wires in a control model in which the movable portion is driven by a pair of opposed motors with use of the wires, and controls the pair of motors, based on an acceleration reference value obtained from the control system.

### Advantageous Effects of Invention

According to the technology disclosed herein, there can be provided the control apparatus and control method for controlling, independently of the bilateral control system, the parallel wire mechanism while preventing vibration and elongation unique to the wires as well as the master-slave system that includes the master apparatus and the slave apparatus at least one of which includes the parallel wire mechanism and that simultaneously achieves bilateral control and the prevention of wire elongation and vibration in a non-interference manner.

Note that, the effects described herein are merely exemplary, and effects provided by the technology disclosed herein are not limited thereto. Further, in some cases, the technology disclosed herein may also exhibit additional effects other than the effects described above.

Other objects, features, and advantages of the technology disclosed herein will be clarified by a more detailed description based on an embodiment described later and the attached drawings.

### BRIEF DESCRIPTION OF DRAWINGS

FIG. 1 is a diagram illustrating a control apparatus and a control method for controlling a master apparatus or slave apparatus including a parallel wire mechanism, and a master-slave system.

FIG. 2 is a diagram illustrating a configuration example of a parallel wire apparatus 100.

FIG. 3 is a perspective view illustrating a master apparatus.

FIG. 4 is a bird's-eye view illustrating the master apparatus.

FIG. 5 is a diagram illustrating a model of the parallel wire mechanism for one degree of freedom.

FIG. 6 is a diagram illustrating a model of a center of gravity mode obtained by mode decomposition of the model of the parallel wire mechanism illustrated in FIG. 5.

FIG. 7 is a diagram illustrating a model of a relative mode obtained by mode decomposition of the model of the parallel wire mechanism illustrated in FIG. 5.

FIG. 8 is a control block diagram of an entire parallel wire control system.

FIG. 9 is a control block diagram of a motor acceleration control system.

FIG. 10 is a diagram illustrating a configuration example of a center of gravity mode control unit 802.

FIG. 11 is a diagram illustrating a configuration example of a relative mode control unit 803.

FIG. 12 is a control block diagram of a master-slave system of a bilateral control system.

### DESCRIPTION OF EMBODIMENT

Now, an embodiment of the technology disclosed herein is described in detail with reference to the drawings. Note that, the present embodiment is described in the following order.

A. Configuration of Master-slave System

B. Configuration Example of Parallel Wire Mechanism

C. Configuration Example of Master Apparatus to which Parallel Wire Mechanism Is Applied

D. Technical Problem of Master-slave System Including Parallel Wire Mechanism

E. Parallel Wire Mechanism Modeling

F. Configuration of Parallel Wire Control System

G. Motor Acceleration Control System

H. Center of Gravity Mode Acceleration Control System

I. Relative Mode Tension Control System

J. Operation of Entire Parallel Wire Control System

K. Bilateral Control of Master-slave System Including Parallel Wire Mechanism

L. Effects

### A. Configuration of Master-Slave System

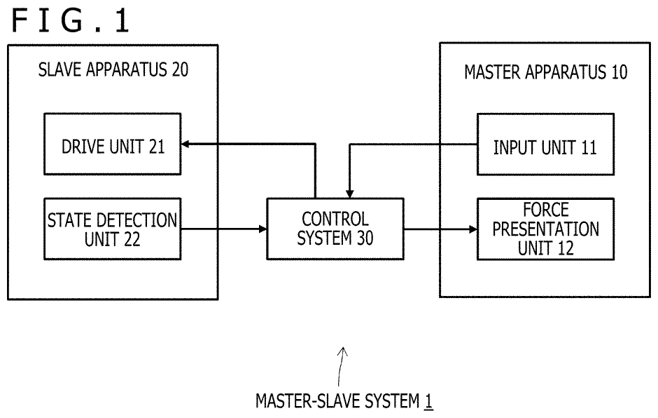

FIG. 1 schematically illustrates a functional configuration example of a master-slave system 1. The master-slave system 1 illustrated in FIG. 1 is a medical robotic system used for implementing, for example, abdominal or thoracic cavity endoscopic surgery. In response to instructions input by a user on the master side through an input apparatus such as a controller, an end effector such as a medical instrument mounted on an arm on the slave side is driven, so that various types of treatment are given on a surgical region of a patient with use of the medical instrument.

The master-slave system 1 illustrated in FIG. 1 includes a master apparatus 10, a slave apparatus 20, and a control system 30 configured to drive the slave apparatus 20 in response to instructions input by the user through the master apparatus 10. When the user operates the master apparatus 10, operation instructions are transmitted to the slave apparatus 20 by wired or wireless communication means through the control system 30, so that the end effector is driven.

The master apparatus 10 includes an input unit 11 configured to allow the user, who is a surgeon or the like, to perform input operation and a force presentation unit 12 configured to present force to the user operating the input unit 11.

The input unit 11 may include, for example, a controller including various input devices such as a lever, a grip, a button, a jog dial, a tactile switch, or a foot pedal switch and a master arm configured to drive the controller. In the present embodiment, at least a part of the master arm may be configured using a parallel wire apparatus.

Further, the force presentation unit 12 includes, for example, a servo motor configured to drive the master arm, a servo motor configured to drive the controller, and the like. The force presentation unit 12 drives, depending on force acting on the end effector on the slave apparatus 20 side, the master arm or the controller to give resistance force to the user operating the controller, for example, thereby presenting the force acting on the end effector such as a medical instrument to the user.

Meanwhile, the slave apparatus 20 includes a slave arm, the end effector mounted on the slave arm, a drive unit 21 configured to drive the slave arm and the end effector, and a state detection unit 22 configured to detect the state of the end effector or slave arm.

The end effector that is mounted on the slave arm includes, for example, a treatment tool that is used by being inserted into the body cavity of a patient in laparoscopic surgery. An openable and closable end effector may be used as the treatment tool. Examples of the openable and closable end effector include jaws, cutting blades, and staplers that generate gripping force. Alternatively, the treatment tool may be a pneumoperitoneum tube, an energy treatment tool, tweezers, a retractor, or the like. The energy treatment tool is a treatment tool for, for example, incising or peeling off a tissue or sealing blood vessels with the action of high-frequency current, ultrasonic vibration, or the like. Further, in the present embodiment, at least a part of the slave arm may be configured using the parallel wire apparatus.

The drive unit 21 includes a motor for operating the slave arm or the end effector. When the motor drives by a control amount calculated by the control system 30, the slave arm or the end effector operates depending on how much the user, who is a surgeon or the like, operates the master arm or the controller.

The state detection unit 22 includes, for example, a sensor configured to detect the position and posture of the slave arm or end effector, a force sensor configured to detect external force acting on the slave arm or the end effector, and the like, and detects the state of the slave arm or the end effector.

The control system 30 achieves, between the master apparatus 10 and the slave apparatus 20, transmission of information associated with the drive control of the slave arm or end effector on the slave apparatus 20 side and with force presentation to the master apparatus 10 side. However, at least one of the slave apparatus 20 or the master apparatus 10 may have the whole or a part of the function of the control system 30. For example, the CPU (Central Processing Unit) (not illustrated) of at least one of the master apparatus 10 or the slave apparatus 20 functions as the control system 30. Alternatively, the CPUs of the master apparatus 10 and the slave apparatus 20 cooperate with each other to function as the control system 30. The control system 30 employs, for example, the bilateral system, and operates the slave apparatus 20 from the master apparatus 10 while feeding back the state of the slave apparatus 20 to the master apparatus 10.

### B. Configuration Example of Parallel Wire Mechanism

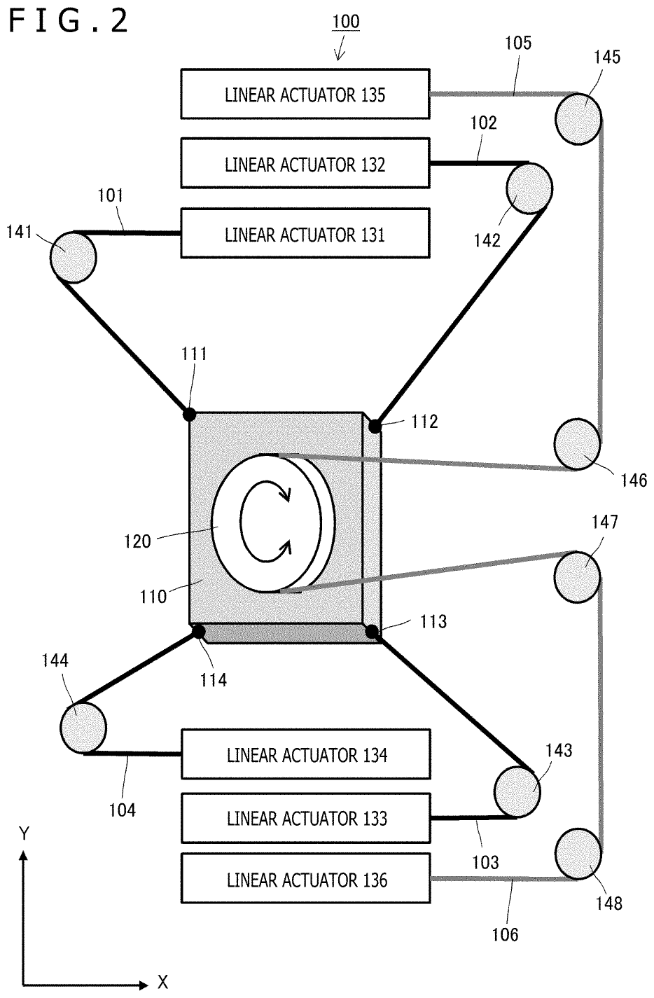

FIG. 2 schematically illustrates a basic configuration example of a parallel wire apparatus 100 that is applied to at least one of the master arm or slave arm, or to a part of at least one of the master arm or slave arm. Here, the plane of the drawing sheet is set as an XY plane, and the direction perpendicular to the plane of the drawing sheet is set as a Z axis. Further, for the purpose of illustration, the parallel wire apparatus 100 illustrated in FIG. 2 has three degrees of freedom in total, namely, translation degrees of freedom in the two XY directions and a rotational degree of freedom around the single Z axis.

The parallel wire apparatus 100 includes six parallel wires 101 to 106 and a movable portion 110 supported by the wires 101 to 106. Further, on the upper surface of the movable portion 110, a rotatable portion 120 is mounted so as to be rotatable at least around the Z axis with respect to the movable portion 110. Note that, it is assumed that the movable portion 110 is slidably supported on a plane such as on a stand (not illustrated).

The wire 101 has a distal end portion fixed to an end portion 111 of the movable portion 110 and a root portion mounted on a linear actuator 131. Through the driving of the linear actuator 131, the length of the wire 101 can be controlled. In the example illustrated in FIG. 2, the distal end portion of the wire 101 is mounted on the end portion 111 while the wire 101 is wound around a direction change pulley 141 to change the driving direction of the linear actuator 131. The distal end portion of the wire 101 is preferably coupled to the end portion 111 with a joint the angle of which is freely changeable, such as a universal joint.

Further, the wire 102 has a distal end portion fixed to an end portion 112 of the movable portion 110 and a root portion mounted on a linear actuator 132. Through the driving of the linear actuator 132, the length of the wire 102 can be controlled. Yet, the distal end portion of the wire 102 is mounted on the end portion 112 while the wire 102 is wound around a direction change pulley 142 to change the driving direction of the linear actuator 132. The distal end portion of the wire 102 is preferably coupled to the end portion 112 with a joint the angle of which is freely changeable, such as a universal joint.

Further, the wire 103 has a distal end portion fixed to an end portion 113 of the movable portion 110 and a root portion mounted on a linear actuator 133. Through the driving of the linear actuator 133, the length of the wire 103 can be controlled. Yet, the distal end portion of the wire 103 is mounted on the end portion 113 while the wire 103 is wound around a direction change pulley 143 to change the driving direction of the linear actuator 133. The distal end portion of the wire 103 is preferably coupled to the end portion 113 with a joint the angle of which is freely changeable, such as a universal joint.

The wire 104 has a distal end portion fixed to an end portion 114 of the movable portion 110 and a root portion mounted on a linear actuator 134. Through the driving of the linear actuator 134, the length of the wire 104 can be controlled. Yet, the distal end portion of the wire 104 is mounted on the end portion 114 while the wire 104 is wound around a direction change pulley 144 to change the driving direction of the linear actuator 134. The distal end portion of the wire 104 is preferably coupled to the end portion 114 with a joint the angle of which is freely changeable, such as a universal joint.

The wire 105 has a distal end portion wound around the side portion of the rotatable portion 120 and fixed. Further, the wire 106 has a distal end portion wound around the side portion of the rotatable portion 120 in a direction opposite to the wire 105 and fixed. Moreover, the wire 105 has a root portion mounted on a linear actuator 135, and the wire 106 has a root portion mounted on a linear actuator 136. Through the driving of the linear actuators 135 and 136, the lengths of the respective wires 105 and 106 can be controlled. Yet, the wire 105 is wound around direction change pulleys 145 and 146 to change the driving direction of the linear actuator 135, and is then wound around the side portion of the rotatable portion 120. Further, the wire 106 is wound around direction change pulleys 147 and 148 to change the driving direction of the linear actuator 136, and is then wound around the side portion of the rotatable portion 120.

The linear actuators 131 to 136 are unitarily controlled by an unillustrated control unit.

The four wires 101 to 104 are parallel wires for translating the movable portion 110 in the XY plane. The control unit synchronously drives the linear actuators 131 to 134 at the root portions of the respective wires 101 to 104 to change the lengths of the wires 101 to 104, and can thus translate the movable portion 110 in the XY plane. Synchronously changing the lengths of the wires 101 to 104 also allows the movable portion 110 to rotate around the Z axis in a certain range of motion on the XYZ coordinate system.

Specifically, the wires 101 and 102 are pulled by the linear actuators 131 and 132, and the wires 103 and 104 are stretched by the linear actuators 133 and 134 so as to be in balance with the pulled wires 101 and 102, thereby moving the movable portion 110 in the positive Y direction. The wires 103 and 104 are pulled by the linear actuators 133 and 134, and the wires 101 and 102 are stretched by the linear actuators 131 and 132 so as to be in balance with the pulled wires 103 and 104, thereby moving the movable portion 110 in the negative Y direction. In a case where the linear actuators are wire winding actuators, the wire is pulled when being wound and stretched when being unwound (the same holds true hereinafter).

Meanwhile, the wires 101 and 104 are pulled by the linear actuators 131 and 134, and the wires 102 and 103 are stretched by the linear actuators 132 and 133 so as to be in balance with the pulled wires 101 and 104, thereby moving the movable portion 110 in the negative X direction. The wires 102 and 103 are pulled by the linear actuators 132 and 133, and the wires 101 and 104 are stretched by the linear actuators 131 and 134 so as to be in balance with the pulled wires 102 and 103, thereby moving the movable portion 110 in the positive X direction.

Further, the two wires 105 and 106 are parallel wires for rotating the rotatable portion 120 on the movable portion 110 around the Z axis. The control unit synchronously drives the linear actuators 135 and 136 to wind one of the two wires 105 and 106 while stretching the other by a length corresponding to the wound amount, and can thus rotate the rotatable portion 120 around the Z axis with respect to the movable portion 110 (or on the XYZ coordinate system).

Specifically, the wire 105 is pulled by the linear actuator 135 and the wire 106 is stretched by the linear actuator 136 so as to be in balance with the pulled wire 105, to thereby allow the rotatable portion 120 to rotate clockwise in FIG. 2. Meanwhile, the wire 106 is pulled by the linear actuator 136 and the wire 105 is stretched by the linear actuator 135 so as to be in balance with the pulled wire 106, to thereby allow the rotatable portion 120 to rotate counterclockwise in FIG. 2.

Note that, changing the lengths of the wires 101 to 104 allows the movable portion 110 not only to translate in the XY plane, but also to rotate around the Z axis to some extent. Thus, the rotation functions of both the movable portion 110 and the rotatable portion 120 are utilized, so that a wider rotational range of motion can be achieved.

In FIG. 2, the wires 101 to 104 arranged in parallel to translate the movable portion 110 are illustrated in black, and the wires 105 and 106 arranged in parallel to rotate the rotatable portion 120 are illustrated in gray.

The linear actuators 131 to 136 can each include, for example, a ball screw, a shaft motor, a linear motor, or a combination of a motor and a gear and rack linear motion structure. However, the linear actuators 131 to 136 are not necessarily required to be the linear actuators as long as the linear actuators 131 to 136 can perform the operation of changing the lengths of the respective wires 101 to 106. For example, the linear actuator can be replaced by a combination of a rotary motor and a mechanism configured to wind the wire with the rotation of the motor.

In the parallel wire apparatus 100 including the four wires 101 to 104 for translation and the two wires 105 and 106 for rotation that are mounted on the movable portion 110 provided with the rotatable portion 120, the arrangement of the wires 101 to 106 and the linear actuators 131 to 136 configured to drive the respective wires 101 to 106 is not limited to the configuration example illustrated in FIG. 2. Further, the number and arrangement of the direction change pulleys 141, 142, etc., around which the respective wires 105 and 106 are wound are not limited to the configuration example illustrated in FIG. 2 either. The arrangement of the linear actuators 131 to 136 and the direction change pulleys 141, 142, etc., may be determined in consideration of, for example, the ranges of motion and ease of operation of the movable portion 110 and the rotatable portion 120, and interference with other unillustrated members in the apparatus 100.

The wires 101 to 106 that are used in the parallel wire apparatus 100 according to the present embodiment can be manufactured using, for example, metal strings (such as stranded stainless steel wire ropes) or chemical fibers. Using metal strings has an advantage that the wires are difficult to elongate. Alternatively, in the case where chemical fibers are used, there is a concern that the wires are easily elongated, but there is an advantage that the wires are flexible. Further, not all the wires to be used are necessarily required to be made of the same material.

Note that, although not illustrated in FIG. 2, the linear actuators 131 to 136 each include an encoder capable of acquiring a position response from the respective linear actuator. Further, the linear actuators 131 to 136 each include detection means such as an encoder capable of acquiring the position of the movable portion 110 and a force sensor capable of acquiring a force response from the movable portion 110.

### C. Configuration Example of Master Apparatus to which Parallel Wire Mechanism is Applied

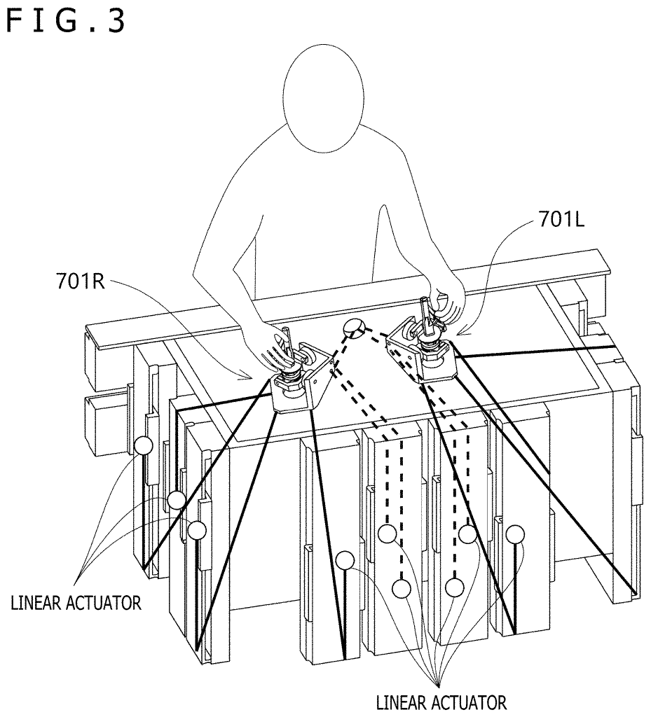

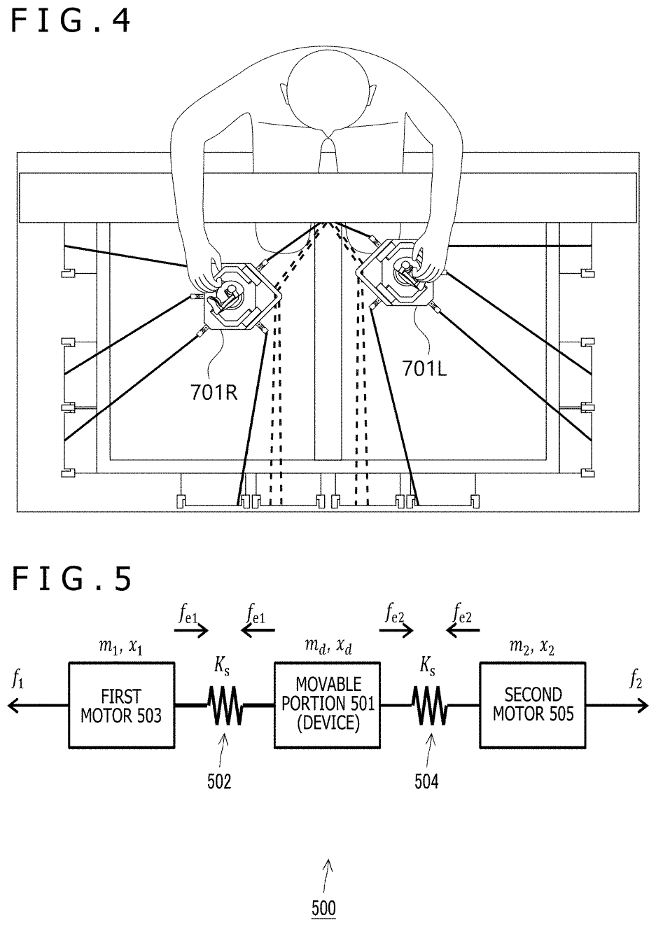

FIG. 3 and FIG. 4 illustrate an example in which the parallel wire system is applied to a master apparatus of a master-slave system. However, FIG. 3 is a perspective view illustrating the master apparatus being operated by the operator when seen from the front of the operator, and FIG. 4 is a bird's-eye view illustrating the master apparatus being operated by the operator. The master apparatus establishes bidirectional communication with an unillustrated slave apparatus. For example, the bilateral system is employed, and the slave apparatus is operated from the master apparatus while the state of the slave apparatus is fed back to the master apparatus.

The main body of the master apparatus is a box-shaped structure with an open upper surface. Plural wires extend in parallel from the side surfaces of the box toward the inside of the box. Moreover, these wires support a left-hand controller 701L and a right-hand controller 701R in the air. Further, plural linear actuators configured to pull the respective wires from the root side are mounted on the master apparatus.

As illustrated in FIG. 3 and FIG. 4, the operator can operate the left-hand controller 701L and the right-hand controller 701R with his/her left and right hands in the box. As described later, the controllers 701L and 701R each have mounted thereon a gripping force sense presentation apparatus, and the operator grips the gripping force sense presentation apparatus with his/her left and right hands to operate the controllers 701L and 701R. The controllers 701L and 701R do not include any power source such as linear actuators for pulling wires. The controllers 701L and 701R can thus be configured to be compact and light and easily be operated by the operator. The left-hand controller 701L and the right-hand controller 701R basically have symmetrical shapes and structures. In the following, description will be given by collectively referring to the controllers 701L and 701R as a “right-hand controller 701.”

The controller 701 includes a controller main body configured to translate in a three-dimensional space with the parallel wires; and a rotatable portion that is mounted on the controller main body to be rotatable around at least one axis and that rotates with the parallel wires. In other words, the parallel wires that support the controller 701 in the air include the parallel wires that translate the controller main body and the parallel wires that rotate the rotatable portion. Further, in FIG. 3 and FIG. 4, the wires used for translating the main body of the controller 701 are illustrated as the solid lines, and the wires used for rotating the rotatable portion in the main body are illustrated as the broken lines. Yet, the details of the mechanism for translating and rotating the controller 701 are described later. The wires are pulled by the respective linear actuators at the root portions.

The root portion of each wire faces the translation direction of the corresponding linear actuator. Meanwhile, the distal end portion of each wire faces the movement direction of the object to be translated or rotated. The wires are each wound around one or two or more pulleys (not illustrated in FIG. 3 and FIG. 4) on the way, to be appropriately folded or reoriented, and are arranged so as not to interfere with each other.

The wires that are used in the master apparatus can be manufactured using, for example, metal strings (such as stranded stainless steel wire ropes) or chemical fibers. Using metal strings has an advantage that the wires are difficult to elongate. Alternatively, in the case where chemical fibers are used, there is a concern that the wires are easily elongated, but there is an advantage that the wires are flexible. Further, not all the wires to be used are necessarily required to be made of the same material. It is generally assumed that a tension of up to approximately 10 kgf acts on the wires. However, the maximum instantaneous tension may exceed the individual values.

### D. Technical Problem of Master-Slave System Including Parallel Wire Mechanism

It is expected that applying the parallel wire apparatus to at least a part of the master arm or slave arm achieves a reduction in weight and an expansion in range of motion.

Further, in the present embodiment, it is assumed that, as the control system for the master-slave system, the bilateral system in which the slave apparatus is operated from the master apparatus while the state of the slave apparatus is fed back to the master apparatus is applied.

To achieve the bilateral control system, highly accurate simultaneous control of position and force is required. However, in the case where the master-slave system is configured using the parallel wire apparatus, there is a problem that the control accuracy is deteriorated due to vibration or elongation unique to the wires. Further, the bilateral control system and the wire tension control system are not independent of each other, so that there is a concern that the prevention of wire vibration and elongation interferes with the bilateral control system.

Accordingly, herein, a technology for preventing elongation and vibration unique to the wires of the bilateral control system in the master apparatus or slave apparatus including the parallel wire mechanism is proposed below. Further, herein, a technology for simultaneously achieving bilateral control and prevention of wire elongation and vibration in a non-interference manner is proposed below.

### E. Parallel Wire Mechanism Modeling

FIG. 2 illustrates the configuration example of the parallel wire apparatus 100 having the three degrees of freedom, and FIG. 3 and FIG. 4 illustrate the appearance of the master apparatus including the parallel wire mechanism. Here, for simplification of the description, the parallel wire mechanism is modeled for one degree of freedom as illustrated in FIG. 5. It should be understood that the parallel wire mechanism may similarly be designed in terms of the remaining axes.

A one-degree-of-freedom model 500 illustrated in FIG. 5 includes a movable portion 501 (device) at the center, a wire 502 for pulling the movable portion 501 to the left on the drawing sheet, a first motor 503 for applying pulling force to the wire 502, a wire 504 for pulling the movable portion 501 to the right on the drawing sheet, and a second motor 505 for applying pulling force to the wire 504.

Note that, although not illustrated in FIG. 5, the first motor 503 and the second motor 505 each include an encoder capable of acquiring a position response from the respective motor. Further, the first motor 503 and the second motor 505 each include detection means such as an encoder capable of acquiring the position of the movable portion 501 and a force sensor capable of acquiring a force response from the movable portion 501.

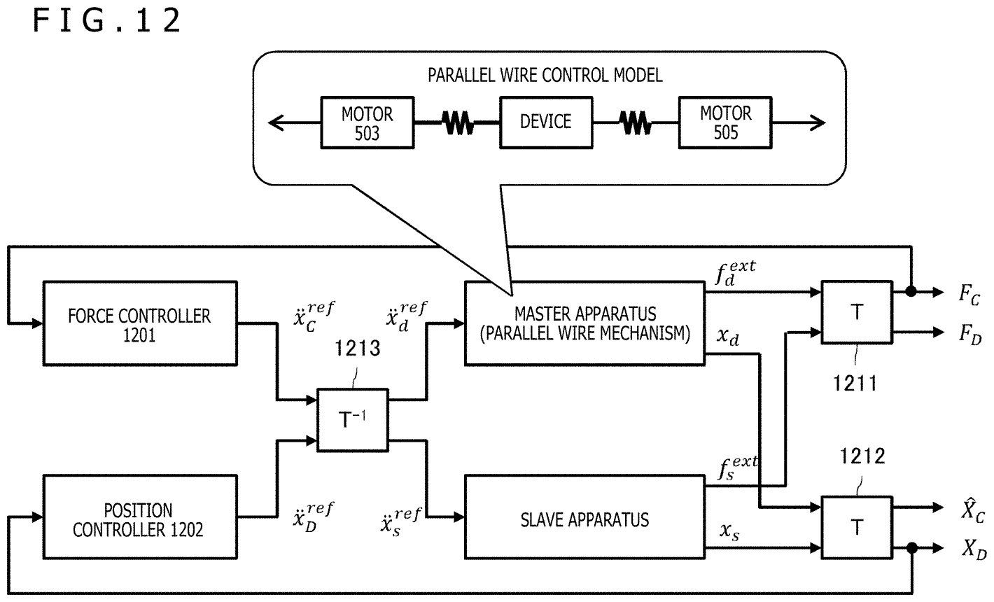

FIG. 12 is a control block diagram illustrating a master-slave system of the bilateral control system. In a case where the master apparatus includes the parallel wire mechanism (for example, see FIG. 3 and FIG. 4), the control model of the parallel wire mechanism as illustrated in FIG. 5 exists in the block of the master apparatus. Yet, the details of the master-slave system illustrated in FIG. 12 are described later. Further, also in a case where the slave apparatus includes the parallel wire mechanism, the control model as illustrated in FIG. 5 exists in the block of the slave apparatus. There is a concern that the bilateral control accuracy is deteriorated due to wire vibration or elongation in the parallel wire mechanism.

Accordingly, herein, a technology for compensating for the elongation and vibration of the wire 502 and the wire 504 in controlling the acceleration of the movable portion 501 with the first motor 503 and the second motor 505 is proposed below.

The wire 502 and the wire 504 are elongated when receiving pulling force from the first motor 503 and the second motor 505, respectively, and are thus modeled as springs. Here, for simplification of the description, the wire 502 and the wire 504 have the same spring constant, which is indicated by Kₛ.

The mass and position of the movable portion 501 are indicated by m_{d} and x_{d}, respectively. Further, the mass and position of the first motor 503 are indicated by m₁ and x₁, respectively. In a similar manner, the mass and position of the second motor 505 are indicated by m₂ and x₂, respectively.

When generating a pulling force f₁ in the left direction on the drawing sheet, the first motor 503 receives an elastic force fₑ₁ in the right direction on the drawing sheet from the wire 502. At this time, the movable portion 501 receives the elastic force fₑ₁ in the left direction on the drawing sheet from the wire 502.

Further, when generating a pulling force f₂ in the right direction on the drawing sheet, the second motor 505 receives an elastic force fₑ₂ in the left direction on the drawing sheet from the wire 504. At this time, the movable portion 501 receives the elastic force fₑ₂ in the right direction on the drawing sheet from the wire 504. The equations of motion of the parallel wire system at this time are expressed as Equations (1) to (5) below.

[Math. 1]

m₁\ddot{x}₁=f₁−fₑ₁−f₁^{dis} (1)

[Math. 2]

m₂\ddot{x}₂=f₂−fₑ₂−f₂^{dis} (2)

[Math. 3]

m_{d}\ddot{x}_{d}=fₑ₁+fₑ₂−f_{d}^{ext}−f_{d}^{dis} (3)

[Math. 4]

fₑ₁=Kₛ(x₁−x_{d}) (4)

[Math. 5]

fₑ₂=Kₛ(x₂−x_{d}) (5)

However, in Equations (1) to (5), f_{d}^{ext} indicates an external force that acts on the movable portion 501, and f_{d}^{dis} indicates a disturbance other than an external force that acts on the movable portion 501, such as friction. Further, f₁^{dis} indicates a disturbance that acts on the first motor 503, and f₂^{dis} indicates a disturbance that acts on the second motor 505.

As can be recognized from Equation (3), in the control model illustrated in FIG. 5, the movable portion 501 is driven with the elastic force from the first motor 503 and the second motor 505. In such a control model, it is difficult to independently control the acceleration response from the movable portion 501 and the tension of the wire 502 and the wire 504.

Accordingly, in the technology proposed herein, to configure control systems configured to independently control the acceleration response from the movable portion 501 and the tension of the wire 502 and the wire 504, the control model illustrated in FIG. 5 is decomposed to a center of gravity mode and a relative mode with the first motor 503 and the second motor 505, by mode decomposition. In the center of gravity mode, a motor C is controlled to make the movable portion 501 achieve desired acceleration. Meanwhile, in the relative mode, a motor R is controlled to achieve a constant elastic force. The center of gravity mode and the relative mode are independent control systems each including only one motor.

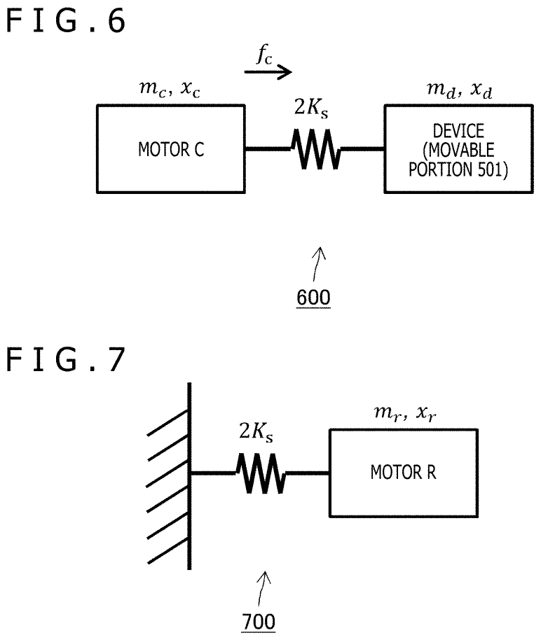

FIG. 6 conceptually illustrates a center of gravity mode 600 of the parallel wire mechanism model. In the center of gravity mode 600, the device (movable portion 501) is coupled to the motor C through a spring having a spring constant 2Kₛ. In the following, the mass and position of the motor C are indicated by m_{c} and x_{c}, respectively, and an elastic force that acts from the spring on the motor C is indicated by f_{c}. From FIG. 6 and Equation (3), when the disturbance is ignored in Equation (5), an equation of motion in the center of gravity mode 600 can be expressed as Equation (6). Thus, from Equation (6), the center of gravity mode 600 can be regarded as a physical model of the two mass point system in which the motor C and the device are connected to each other by the spring. The spring that connects the motor C and the device to each other has the spring constant 2Kₛ that is the sum of the spring constants of the wire 502 and the wire 504.

$$\begin{matrix}[ Math . 6 ] & \\ \begin{matrix}{m}_{d} {\ddot{x}}_{d} = {K}_{s} ( {x}_{1} - {x}_{d} ) + {K}_{s} ( {x}_{2} - {x}_{d} ) \\ = 2 {K}_{s} \frac{{x}_{1} + {x}_{2}}{2} - 2 {K}_{s} {x}_{d} \\ = 2 {K}_{s} ( {x}_{c} - {x}_{d} )\end{matrix} & ( 6 )\end{matrix}$$

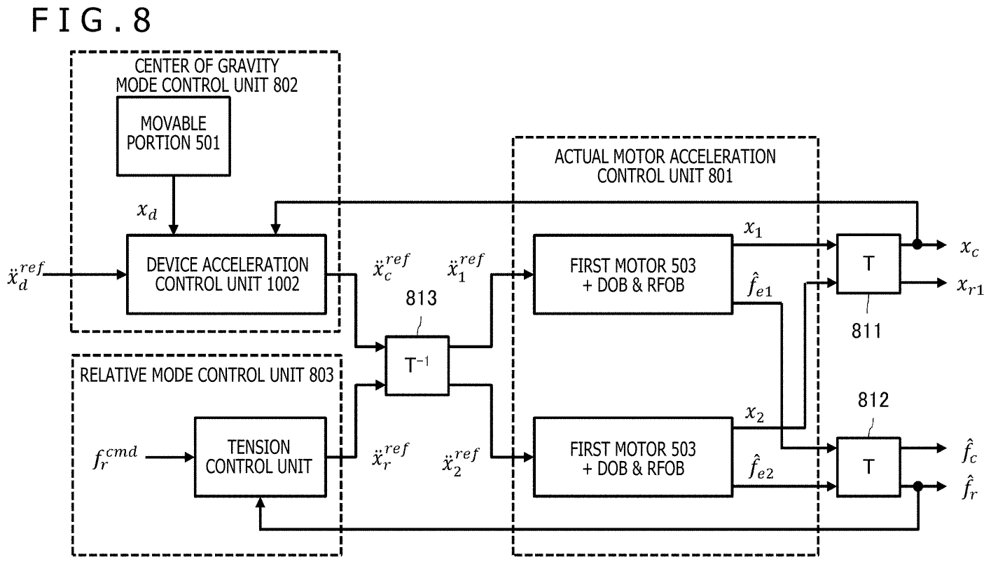

Further, FIG. 7 conceptually illustrates a relative mode 700 of the parallel wire mechanism model. In the relative mode 700, one end of a spring whose opposite end is mounted on a certain wall is pulled by the motor R. The motor R pulls the spring having the spring constant 2Kₛ. In the following, the mass and position of the motor R are indicated by mᵣ and xᵣ, respectively, and an elastic force that acts on the device (movable portion 501) through the spring is indicated by fᵣ. From FIG. 7 and Equations (1) and (2), the static characteristics of the relative mode can be expressed as Equation (7). Thus, also from Equation (7), the relative mode 700 can be regarded as a physical model in which the motor R pulls the spring with a resultant force of force generated by the first motor 503 and force generated by the second motor 505 (−f₁+f₂). The spring that is pulled by the motor R has the spring constant 2Kₛ that is the sum of the spring constants of the wire 502 and the wire 504.

$$\begin{matrix}[ Math . 7 ] & \\ \begin{matrix}0 = {f}_{1} - {f}_{e 1} - ( {f}_{2} - {f}_{e 2} ) \\ = 2 {K}_{s} \frac{{x}_{1} + {x}_{2}}{2} - ( - {f}_{1} + {f}_{2} ) \\ = 2 {K}_{s} {x}_{r} - ( - {f}_{1} - {f}_{2} )\end{matrix} & ( 7 )\end{matrix}$$

Here, the kinematics of the motor C in the center of gravity mode can be expressed as the center-of-gravity motion of the first motor 503 and the second motor 505. Thus, in the center of gravity mode, the position x_{c} and the elastic force f_{c} of the motor C are expressed as Equation (8) and Equation (9), respectively.

[Math. 8]

x_{c}=½(x₁+x₂) (8)

[Math. 9]

f_{c}=½(fₑ₁+fₑ₂) (9)

Further, the kinematics of the motor R in the relative mode can be expressed as the relative motion of the first motor 503 and the second motor 505. Thus, in the relative mode, the position xᵣ and the elastic force fᵣ of the motor R are expressed as Equation (10) and Equation (11), respectively.

[Math. 10]

xᵣ=½(−x₁+x₂) (10)

[Math. 11]

fᵣ=½(−fₑ₁+fₑ₂) (11)

Thus, the control objectives of the center of gravity mode and the relative mode in parallel wire driving can be set independently as follows.

Center of gravity mode: Controlling the motor C to make the device achieve given acceleration.

Relative mode: Controlling the motor R to make fᵣ always converge to a certain value.

That is, in the center of gravity mode, the control objective is to drive the motor C and control the device without vibration generation and spring elongation (device acceleration control). Further, in the relative mode, the control objective is to drive the motor R and pull the spring with a constant force (constant tension control).

The applicant of the present invention considers that, by individually designing appropriate control systems for the center of gravity mode and relative mode described above, it is possible to prevent wire vibration and elongation by the wire tension control system independent of the bilateral control system and to allow the bilateral control system to achieve highly accurate simultaneous control of position and force.

Here, Equations (8) to (11) can be combined to obtain Equations (12) to (14).

$$\begin{matrix}[ Math . 12 ] & \\ [ \begin{matrix}{x}_{c} \\ {x}_{r}\end{matrix} ] = T [ \begin{matrix}{x}_{1} \\ {x}_{2}\end{matrix} ] & ( 12 ) \\ [ Math . 13 ] & \\ [ \begin{matrix}{f}_{c} \\ {f}_{r}\end{matrix} ] = T [ \begin{matrix}{f}_{e 1} \\ {f}_{e 2}\end{matrix} ] & ( 13 ) \\ [ Math . 14 ] & \\ T = \frac{1}{2} [ \begin{matrix}1 & 1 \\ - 1 & 1\end{matrix} ] & ( 14 )\end{matrix}$$

In Equations (12) to (14), T indicates a transformation matrix for transforming the motor space of the first motor 503 and the second motor 505, which actually exist, to the mode space of the motor C in the center of gravity mode and the motor R in the relative mode, by coordinate transformation. The row vectors in the first and second rows of T described in Equation (14) are orthogonal to each other. Thus, the center of gravity mode and the relative mode can independently configure the control systems without interfering with each other. Note that, with the use of an inverse matrix T⁻¹ for T, the mode space can be transformed to the motor space by coordinate transformation (which is described later).

### F. Configuration of Parallel Wire Control System

FIG. 8 is a control block diagram illustrating an entire parallel wire control system. Note that, in FIG. 8, the variables with the circumflexes (or hat symbols) mean the estimated values of the variables in question. Further, the variables with the double dots mean the acceleration values of the variables in question (the same holds true hereinafter).

The control block diagram of FIG. 8 includes an actual motor acceleration control unit 801 configured to control the acceleration of the first motor 503 and the second motor 505, a center of gravity mode control unit 802 configured to control the first motor 503 and the second motor 505 in the center of gravity mode, and a relative mode control unit 803 configured to control the first motor 503 and the second motor 505 in the relative mode.

The control objective of the center of gravity mode control unit 802 is to drive the motor C and control the acceleration of the device (movable portion 501) without spring vibration and spring elongation. The center of gravity mode control unit 802 determines the acceleration reference value for the motor C on the basis of the externally given acceleration reference value for the device (movable portion 501). Further, the control objective of the relative mode control unit 803 is to pull the device (movable portion 501) with a constant elastic force. The relative mode control unit 803 determines the acceleration reference value for the motor R on the basis of a tension fᵣ^{cmd} determined in advance. The center of gravity mode control unit 802 and the relative mode control unit 803 are configured as independent control systems.

The acceleration reference value for the motor C determined by the center of gravity mode control unit 802 and the acceleration reference value for the motor R determined by the relative mode control unit 803 are subjected to coordinate transformation from the mode space to the motor space by a transformation unit 813 with use of the inverse matrix T⁻¹, and are given to the actual motor acceleration control unit 801 as the acceleration reference value for the first motor 503 and the acceleration reference value for the second motor 505.

The actual motor acceleration control unit 801 controls the first motor 503 and the second motor 505 on the basis of the respective acceleration reference values given through the transformation unit 813. The first motor 503 and the second motor 505 each include an encoder capable of acquiring a position response and a force sensor capable of acquiring a force response.

The position x₁ of the first motor 503 and the position x₂ of the second motor 505 are subjected to coordinate transformation from the motor space to the mode space by a transformation unit 811 with use of the matrix T, and the obtained position x_{c} of the motor C is fed back from the actual motor acceleration control unit 801 to the center of gravity mode control unit 802, so that a loop for controlling the acceleration of the device (movable portion 501) is formed.

Further, the estimated value of the elastic force fₑ₁ generated in the wire 502 when the first motor 503 drives and the estimated value of the elastic force fₑ₂ generated in the wire 504 when the second motor 505 drives are subjected to coordinate transformation from the motor space to the mode space by a transformation unit 812 with use of the matrix T. The obtained estimated value of the elastic force fᵣ of the motor R is fed back from the actual motor acceleration control unit 801 to the relative mode control unit 803, so that a loop for controlling the spring to have a constant tension is formed.

### G. Motor Acceleration Control System

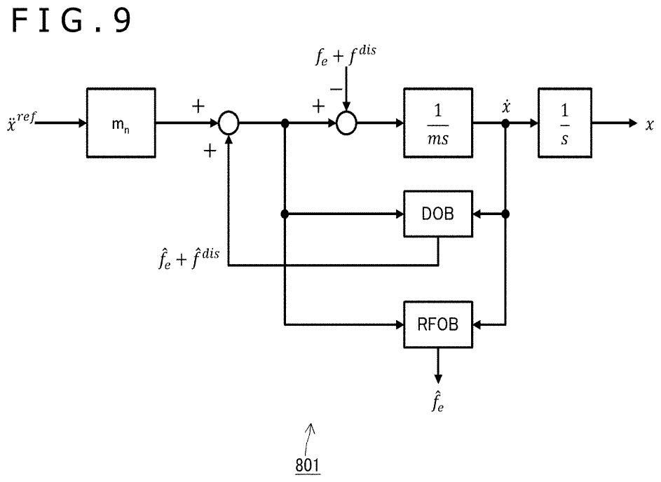

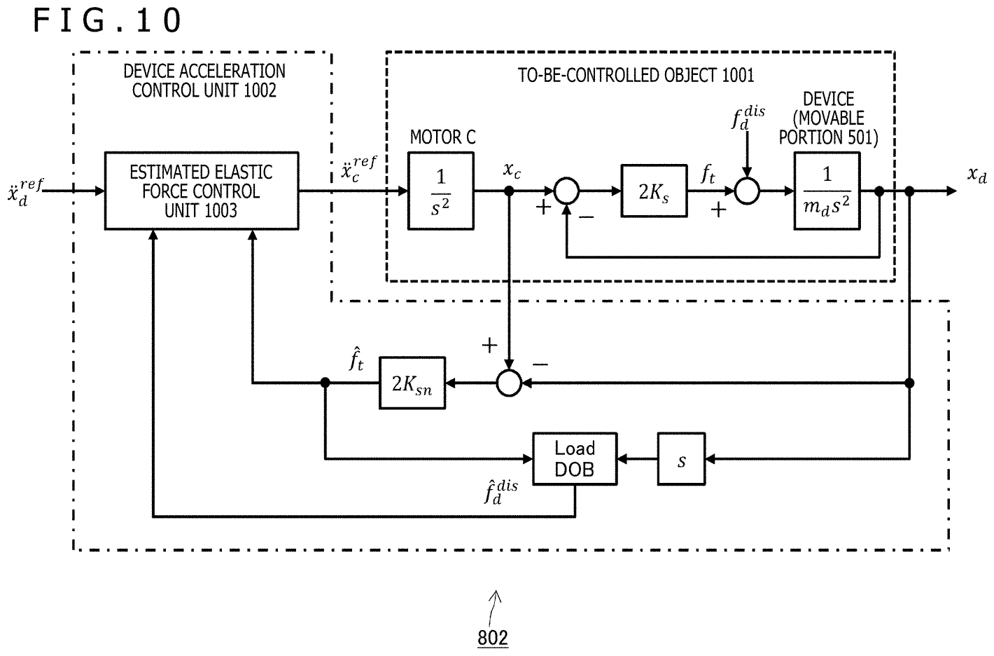

Next, the actual motor acceleration control unit 801 is described in detail. FIG. 9 is a control block diagram illustrating a motor acceleration control system that is applicable to the first motor 503 and the second motor 505. However, s in FIG. 9 indicates the Laplace operator (the same holds true hereinafter). As illustrated in FIG. 9, robust acceleration control is performed with the first motor 503 and the second motor 505 each having mounted thereon a disturbance observer (DOB). Further, to estimate the elastic force fₑ₁ and the elastic force fₑ₂ that are applied to the first motor 503 and the second motor 505, respectively, the first motor 503 and the second motor 505 each also have mounted thereon a reaction force estimation observer (Reaction Force Observer: RFOB).

A force that is the sum of a disturbance f^{dis} and an elastic force fₑ obtained by multiplying an externally input acceleration reference value by a mass nominal value mₙ acts on a to-be-controlled object to displace the to-be-controlled object to a position x. The disturbance observer (DOB) estimates, from the elastic force fₑ and the speed of the to-be-controlled object, the elastic force fₑ and the disturbance f^{dis}, and feeds back the elastic force fₑ and the disturbance f^{dis} to the to-be-controlled object as input. Further, the reaction force estimation observer (RFOB) performs estimation with the elastic force fₑ and the speed of the to-be-controlled object, and externally outputs the result.

### H. Center of Gravity Mode Acceleration Control System

Next, the center of gravity mode control unit 802 is described in detail. As already described, the center of gravity mode can be expressed as the physical model of the two mass point system in which the device (movable portion 501) is coupled to the motor C. The control objective of the center of gravity mode control unit 802 is to drive the motor C and control the acceleration of the device (movable portion 501) without spring vibration and spring elongation.

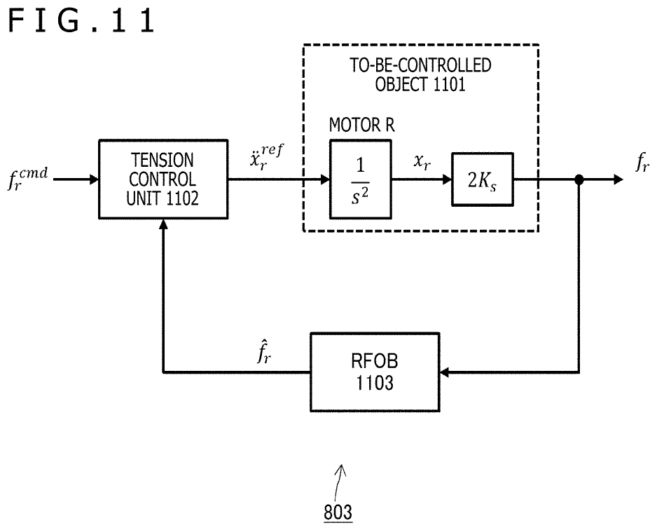

FIG. 10 conceptually illustrates a configuration example of the center of gravity mode control unit 802. The center of gravity mode control unit 802 includes a to-be-controlled object 1001 and a device acceleration control unit 1002 configured to control the acceleration of the motor C in the to-be-controlled object 1001.

The to-be-controlled object 1001 is a physical model of the two mass point system corresponding to the parallel wire mechanism model in the center of gravity mode illustrated in FIG. 6, and includes the motor C, a spring having the elastic coefficient 2Kₛ, and the device (movable portion 501). The device acceleration control unit 1002 receives the acceleration reference value for the device (movable portion 501), which is any value, and gives the acceleration reference value for the motor C in the center of gravity mode. In a case where the parallel wire mechanism is incorporated in a bilateral master-slave system, the acceleration reference value for the device (movable portion 501) is given from the system in question.

The equations of motion of the first motor 503, the second motor 505, and the movable portion 501 (that is, actual physical systems) are expressed as Equations (1) to (3). When these equations of motion are subjected to mode transformation (however, the disturbance is ignored), the equation of motion of the device (movable portion 501) can be rewritten as Equation (6). The object of the device acceleration control unit 1002 is to control the acceleration of the device on the left side on the first line in Equation (6).

Here, the left side on the first line in Equation (6) is an elastic force fₜ in what is generally called Hooke's law. Thus, it can be recognized that the acceleration of the device (movable portion 501) may be controlled to a given value through the appropriate control of 2Kₛ (x_{c}−x_{d}) and the modeled elastic force fₜ. Accordingly, Equation (6) is rewritten as Equation (15).

[Math. 15]

\hat{f}ₜ=2Kₛₙ(x_{c}−x_{d}) (15)

From Equation (15), it can be recognized that the feedback control system configured to achieve the desired elastic force fₜ may be configured through the estimation of the elastic force fₜ with use of the position x_{c} of the motor C in the center of gravity mode, the position x_{d} of the device (movable portion 501), and a spring constant nominal value Kₛₙ. Through the appropriate control of the desired elastic force fₜ, the control system configured to prevent the vibration and elongation of the wire 502 and the wire 504 can be achieved.

Further, it is assumed that, due to a force other than an external force on the device (movable portion 501), such as friction, the disturbance f_{d}^{dis} is generated, for example, the spring constant is deviated from the nominal value Kₛₙ. In the configuration example illustrated in FIG. 10, similarly to the motors 503 and 505, the disturbance f_{d}^{dis} can be compensated for by a disturbance observer using an estimated elastic force and the speed of the device.

Then, the device acceleration control unit 1002 calculates, in consideration of the estimated elastic force and the estimated value of the disturbance f_{d}^{dis} that acts on the device, the acceleration reference value for the motor C in the center of gravity mode from the acceleration reference value for the device (movable portion 501), which is any value, and outputs the acceleration reference value to the to-be-controlled object 1001. The motor C in the center of gravity mode is expressed as the double integral in FIG. 10 on the assumption that each motor has mounted thereon a disturbance observer configured to compensate for load disturbance (Load DOB) and optimal acceleration control is thus achieved.

With reference to FIG. 10, in the to-be-controlled object 1001, the motor C is driven on the basis of the acceleration reference value for the motor C given from the device acceleration control unit 1002, so that the motor C is displaced to the position x_{c}. As a result, the elastic force fₜ (=2Kₛ (x_{c}−x_{d})) and the disturbance f_{d}^{dis} act on the device (movable portion 501) through the spring 2Kₛ having the spring constant, so that the position x_{d} of the device is shifted. In the device acceleration control unit 1002, an elastic force that acts on the device is estimated using a spring constant nominal value 2Kₛₙ. The disturbance observer (DOB) estimates, using the estimated elastic force and the speed of the device, the disturbance f_{d}^{dis} that acts on the device. Then, the estimated elastic force control unit 1003 calculates, in consideration of the estimated elastic force and the estimated value of the disturbance f_{d}^{dis} that acts on the device, the acceleration reference value for the motor C in the center of gravity mode from the acceleration reference value for the device (movable portion 501), which is any value, and outputs the acceleration reference value to the to-be-controlled object 1001.

Note that, the substantial function of the device acceleration control unit 1002 can be implemented using a general computer such as a personal computer.

### I. Relative Mode Tension Control System

Next, the relative mode control unit 803 is described. As already described, the relative mode can be expressed as the physical model in which one end of the spring whose opposite end is mounted on a certain wall is pulled (see FIG. 7). The control objective of the relative mode control unit 803 is to pull the device (movable portion 501) with a constant elastic force.

FIG. 11 conceptually illustrates a configuration example of the relative mode control unit 803. The relative mode control unit 803 includes a to-be-controlled object 1101 including a spring having the spring constant 2Kₛ and a tension controller 1102 configured to control the tension of the spring in the to-be-controlled object 1101. In addition, the elastic force fᵣ that acts from the spring on the motor R in the to-be-controlled object 1101 is fed back to the tension controller 1102, so that the force control system that achieves the tension fᵣ following a certain constant tension command value fᵣ^{cmd} for the spring having the spring constant 2Kₛ is configured. Further, the motor R has mounted thereon a reaction force estimation observer (RFOB) 1103, so that an estimated elastic force in the relative mode is calculated from an estimated elastic force on each motor as in Equation (16).

[Math. 16]

\hat{f}ᵣ=½(\hat{f}ₑ₁+\hat{f}ₑ₂) (16)

The tension controller 1102 calculates, from the estimated tension described in Equation (16), the acceleration reference value for the motor R in the relative mode.

Referring to FIG. 11, in the to-be-controlled object 1101, the motor R drives on the basis of the acceleration reference value for the motor R given from the tension control unit 1102, so that the motor R is displaced to the position xᵣ. As a result, the elastic force fᵣ acts on the spring 2Kₛ having the spring constant. The reaction force estimation observer (RFOB) 1103 estimates the elastic force fᵣ. Then, the tension control unit 1102 calculates the acceleration reference value for the motor R so that the estimated elastic force follows the tension fᵣ^{cmd} determined in advance, and outputs the acceleration reference value to the to-be-controlled object 1101.

### J. Operation of Entire Parallel Wire Control System

Referring to FIG. 8 again, the entire parallel wire control system is described. As described above, the center of gravity mode control unit 802 outputs the acceleration reference value for the motor C for making the device (movable portion 501) achieve given acceleration. Further, the relative mode control unit 803 outputs the acceleration reference value for the motor R for making the elastic force fᵣ caused by the motor R always converge a certain value.

On the basis of Equation (12), as described in Equation (17), the mode space including the acceleration reference values for the motor C and the motor R can be transformed to the motor space including the acceleration reference values for the first motor 503 and the second motor 505, with use of the inverse matrix T⁻¹. Then, the actual motor control unit 801 controls the acceleration of the first motor 503 and the second motor 505 on the basis of the obtained acceleration target values.

$$\begin{matrix}[ Math . 17 ] & \\ [ \begin{matrix}{\ddot{x}}_{1}^{ref} \\ {\ddot{x}}_{2}^{ref}\end{matrix} ] = {T}^{- 1} [ \begin{matrix}{\ddot{x}}_{c}^{ref} \\ {\ddot{x}}_{r}^{ref}\end{matrix} ] & ( 17 )\end{matrix}$$

The center of gravity mode control unit 802 and the relative mode control unit 803 are configured as the independent control systems, and can thus independently set the acceleration reference value for the motor C and the acceleration reference value for the motor R, respectively. As already described, the transformation matrix T for achieving coordinate transformation from the motor space to the mode space has, on the first and second rows, the row vectors orthogonal to each other as described in Equation (14), so that the center of gravity mode and the relative mode can independently configure the control systems without interfering with each other. Thus, the actual motor control unit 801 controls, on the basis of the acceleration reference values obtained by coordinate transformation with Equation (17), the first motor 503 and the second motor 505, and is thus able to prevent wire vibration and elongation and achieve robust parallel wire control without interfering with the parallel wire control system.

### K. Bilateral Control of Master-Slave System Including Parallel Wire Mechanism

In the case of the parallel wire mechanism incorporated in the bilateral master-slave system, the prevention of wire vibration and elongation in the parallel wire mechanism and highly accurate bilateral control can be achieved without interference with the bilateral control system only by giving the parallel wire control system (see FIG. 8) a desired acceleration reference value for the device (movable portion 501).

FIG. 12 is a control block diagram illustrating the master-slave system of the bilateral control system. In the present embodiment, it is assumed that the parallel wire mechanism exists in the block of the master apparatus and that the parallel wire control system (see FIG. 8) described in Item C is applied. With the parallel wire control system illustrated in FIG. 8, through the input of the acceleration reference value for the device (movable portion 501), the tension of the wire 502 and the wire 504 can be kept constant and wire vibration and elongation can be prevented.

In the case where the parallel wire mechanism is used in the master apparatus, the control objectives of bilateral control can be expressed as Equations (18) and (19).

[Math. 18]

f_{d}+fₛ=0∈Rⁿ (18)

[Math. 19]

x_{d}−xₛ=0∈Rⁿ (19)

In Equations (18) and (19), f∈Rⁿ and x∈Rⁿ indicate a force response vector and a position response vector, respectively. Note that, Rⁿ indicates a real coordinate space of n dimensions. Further, the subscript d indicates the movable portion 501 of the parallel wire mechanism incorporated in the master apparatus, and the subscript s indicates the slave apparatus. Equation (18) represents the control objective related to the law of action and reaction in terms of force between the master apparatus and the slave apparatus, and means making the sum of the force of the movable portion 501 (master apparatus) and the force of the slave apparatus zero (common mode). Further, Equation (19) represents the control objective related to the position followability between the master apparatus and the slave apparatus, and means making a difference in position between the movable portion 501 (master apparatus) and the slave apparatus zero (differential mode).

To achieve the two control objectives described in Equations (18) and (19), the force response vectors and position response vectors of the master apparatus (parallel wire mechanism) and the slave apparatus described in Equation (22) are transformed to the mode space with use of a transformation matrix T∈R^{n×n}, according to Equations (20) and (21) by coordinate transformation. Further, the transformation matrix T∈R^{n×n} is described in Equation (22).

$$\begin{matrix}[ Math . 20 ] & \\ [ \begin{matrix}{F}_{C} \\ {F}_{D}\end{matrix} ] = T [ \begin{matrix}{f}_{d} \\ {f}_{s}\end{matrix} ] & ( 20 ) \\ [ Math . 21 ] & \\ [ \begin{matrix}{X}_{C} \\ {X}_{D}\end{matrix} ] = T [ \begin{matrix}{x}_{d} \\ {x}_{s}\end{matrix} ] & ( 21 ) \\ [ Math . 22 ] & \\ T = [ \begin{matrix}{I}_{n} & {I}_{n} \\ {I}_{n} & - {I}_{n}\end{matrix} ] & ( 22 )\end{matrix}$$

In Equations (20) and (21), F∈Rⁿ and X∈Rⁿ indicate the force response vector in the mode space and the position response vector therein, respectively. Further, the subscript C indicates the common mode and the subscript D indicates the differential mode. Further, in Equation (22), Iₙ indicates an identity matrix of n dimensions.

In FIG. 12, a transformation unit 1211 transforms force response vectors (f_{d}^{ext} and fₛ^{ext}) of the master apparatus (parallel wire mechanism) and the slave apparatus to the mode space by coordinate transformation, and feeds back a force response F_{c} in the common mode to a force controller 1201. Further, a transformation unit 1212 transforms position response vectors (x_{d}^{ext} and xₛ^{ext}) of the master apparatus (parallel wire mechanism) and the slave apparatus to the mode space, and feeds back a position response X_{D} in the differential mode to a position controller 1202.

At this time, the control objectives of bilateral control can be rewritten as Equations (23) and (24).

[Math. 23]

F_{C}=0∈Rⁿ (23)

[Math. 24]

x_{D}=0∈Rⁿ (24)

Thus, the force control system and the position control system in the mode space are described as Equation (25) and Equation (26), respectively.

[Math. 25]

\ddot{X}_{C}^{ref}^{_{f}}=C_{f}(0−F_{C})=−C_{f}F_{C} (25)

Note that, \ddot{X}_{C}^{ref}∈Rⁿ is the acceleration reference vector in the common mode and C_{f} is the force controller in the mode space.

[Math. 26]

\ddot{X}_{D}^{ref}=Cₚ(0−X_{D})=−CₚF_{D} (26)

Note that, \ddot{X}_{D}^{ref}∈Rⁿ is the acceleration reference vector in the differential mode and Cₚ is the position controller in the mode space.

In FIG. 12, the force controller 1201 calculates an acceleration reference value in the common mode from the fed-back force response F_{c} in the common mode according to Equation (25). Further, the position controller 1202 calculates an acceleration reference value in the differential mode from the fed back position response X_{D} in the differential mode according to Equation (26).

As described in Equation (27), through the inverse transformation of the acceleration reference vectors in the mode space, the acceleration reference vectors of the master apparatus and the slave apparatus are obtained.

$$\begin{matrix}[ Math . 27 ] & \\ [ \begin{matrix}{\ddot{x}}_{d}^{ref} \\ {\ddot{x}}_{s}^{ref}\end{matrix} ] = {T}^{- 1} [ \begin{matrix}{\ddot{X}}_{C}^{ref} \\ {\ddot{X}}_{D}^{ref}\end{matrix} ] & ( 27 )\end{matrix}$$

In FIG. 12, the acceleration reference vector including the acceleration reference value obtained by the force controller 1201 and the acceleration reference value obtained by the position controller 1202 is transformed by an inverse transformation unit 1213 to the acceleration reference vector including the acceleration reference value for the movable portion 501 and the acceleration reference value for the slave apparatus, by inverse coordinate transformation. Then, the obtained acceleration reference values are input to the master apparatus and the slave apparatus. The acceleration reference value that is input to the master apparatus is the acceleration reference value that is given to the center of gravity mode control unit 802 in the parallel wire control system illustrated in FIG. 8. The center of gravity mode control unit 802 drives the motor C to control the movable portion 501 on the basis of the acceleration while preventing spring vibration and spring elongation. At this time, the center of gravity mode control unit 802 performs the following, with the control objective of making the movable portion 501 achieve acceleration corresponding to this acceleration reference value.

The center of gravity mode control unit 802 performs robust acceleration control in which the disturbance observers are applied to both the master apparatus including the parallel wire mechanism and the slave apparatus, and is thus able to achieve highly accurate bilateral control.

Note that, the embodiment in which the parallel wire mechanism is included in the block of the master apparatus is mainly described herein, but the parallel wire control system described in Item C is also applicable to a case where the parallel wire mechanism is present in the block of the slave apparatus and a case where the parallel wire mechanism is included in each of the master apparatus and the slave apparatus.

### L. Effects

Finally, the effects of the technology disclosed herein are summarized.

According to the technology disclosed herein, highly accurate bilateral control can be achieved in one of the master apparatus or slave apparatus including the parallel wire mechanism, or in both of them.

According to the technology disclosed herein, a wider range of motion can be achieved with the compact and light movable portion of the parallel wire mechanism, by setting the wires to given lengths. With this, excellent operability of the bilateral master-slave system can be achieved.

According to the technology disclosed herein, the vibration phenomenon and elongation unique to the wires of the parallel wire mechanism can be prevented. Highly accurate positioning performance is required especially in medical applications. With the parallel wire mechanism to which the technology disclosed herein is applied, wire vibration and elongation can be prevented so that highly accurate positioning performance can be achieved.

The parallel wire control system to which the technology disclosed herein is applied performs control at the acceleration level, and is thus able to simultaneously achieve the prevention of wire vibration and elongation and wire constant tension control without interfering with the control objectives of the control.

The parallel wire mechanism to which the technology disclosed herein is applied is applicable to bilateral control systems that require highly accurate positioning performance, and can thus be utilized in master-slave systems in various industrial fields including the medical field.

### INDUSTRIAL APPLICABILITY

The technology disclosed herein has been described above in detail with reference to the specific embodiment. However, it is obvious that those skilled in the art can make modifications or substitutions of the embodiment without departing from the gist of the technology disclosed herein.

The technology disclosed herein is mainly applicable to master-slave systems using parallel wires. In particular, when the technology disclosed herein is applied to a bilateral master-slave system, bilateral control and prevention of wire elongation and vibration can be simultaneously achieved in a non-interference manner.

In short, the technology disclosed herein has been described in a form of illustration, and the details described herein should not be interpreted in a limited manner. In order to determine the gist of the technology disclosed herein, the appended claims should be taken into account.

Note that, the technology disclosed herein can also take the following configurations.

(1) A control apparatus for a parallel wire apparatus configured to pull a movable portion with a plurality of wires,

the control apparatus being configured to control force and a position of the movable portion, based on acceleration, while preventing elongation and vibration of the wires.

(1-1) The control apparatus according to Item (1), in which the force and position of the movable portion is controlled while the elongation and vibration of the wires are prevented.

(2) The control apparatus according to Item (1),

in which the control apparatus constitutes a control system configured to independently control an acceleration response and tension of the wires in a control model in which the movable portion is driven by a pair of opposed motors with use of the wires, and controls the pair of motors, based on an acceleration reference value obtained from the control system.

(3) The control apparatus according to Item (2),

in which the control system has a center of gravity mode in which a motor C is controlled to make the movable portion achieve desired acceleration and a relative mode in which a motor R is controlled to make an elastic force that acts on the wires constant, and performs acceleration control on the pair of motors, based on acceleration reference values for the motor C and the motor R.

(4) The control apparatus according to Item (3), in which the center of gravity mode includes a physical model of a two mass point system in which the motor C and the movable portion are connected to each other by a spring.

(5) The control apparatus according to Item (3) or (4), in which kinematics of the motor C is expressed as center-of-gravity motion of the pair of motors.

(6) The control apparatus according to any one of Items (3) to (5), in which the relative mode includes a physical model in which the motor R pulls a spring with a resultant force of force generated by the pair of motors.

(7) The control apparatus according to any one of Items (3) to (6), in which kinematics of the motor R is expressed as relative motion of the pair of motors.

(8) The control apparatus according to any one of Items (3) to (7), in which the acceleration reference value for the motor C determined to make the movable portion achieve the desired acceleration and the acceleration reference value for the motor R determined to make the elastic force constant are subjected to coordinate transformation, to thereby obtain an acceleration reference value for the pair of motors.

(9) The control apparatus according to any one of Items (2) to (8), in which the pair of motors each have mounted thereon a disturbance observer.

(10) The control apparatus according to any one of Items (2) to (9), in which the pair of motors each have mounted thereon a reaction force estimation observer.

(11) The control apparatus according to any one of Items (3) to (10), in which the control system controls, in the center of gravity mode, an estimated elastic force that acts from the motor C on the movable portion, to thereby prevent the elongation and vibration of the wires.

(12) The control apparatus according to Item (11),

in which the control system includes a disturbance observer configured to estimate a disturbance on the movable portion, based on the estimated elastic force that acts from the motor C on the movable portion and a speed of the movable portion, in the center of gravity mode, and obtains the acceleration reference value for the motor C from the desired acceleration of the movable portion in consideration of the estimated elastic force and the disturbance estimated.

(13) The control apparatus according to any one of Items (3) to (12), in which the control system controls, in the relative mode, the motor R so that predetermined tension acts on the wires.

(14) The control apparatus according to Item (13),

in which the control system includes a reaction force estimation observer configured to estimate an elastic force that acts on the wires by the motor R, in the relative mode, and obtains the acceleration reference value for the motor R, based on the predetermined tension and the elastic force estimated.

(15) The control apparatus according to any one of Items (3) to (14), in which the control apparatus performs coordinate transformation on a mode space including the acceleration reference values for the motor C and the motor R in the control system, to thereby obtain an acceleration reference value for the pair of motors.

(16) The control apparatus according to Item (15),

in which positions of the pair of motors in a motor space are transformed to the mode space by coordinate transformation, and a position of the motor C calculated is fed back to the center of gravity mode of the control system, and

force generated by the pair of motors in the motor space is transformed to the mode space by coordinate transformation, and an estimated elastic force of the motor R calculated is fed back to the relative mode of the control system.

(17) A control method for a parallel wire apparatus configured to pull a movable portion with a plurality of wires, the control method including steps of:

controlling, by a control system, a motor C in a center of gravity mode to make the movable portion achieve desired acceleration, the control system being configured to independently control an acceleration response and tension of the wires in a control model in which the movable portion is driven by a pair of opposed motors with use of the wires;

controlling, by the control system, a motor R in a relative mode to make an elastic force that acts on the wires constant; and

performing, by the control system, acceleration control on the pair of motors, based on acceleration reference values for the motor C and the motor R.

(18) A master-slave system including:

a master apparatus and a slave apparatus at least one of which includes a parallel wire mechanism configured to pull a movable portion with a plurality of wires; and

a control apparatus configured to control force and a position of the movable portion, based on acceleration.

(18-1) The master-slave system according to Item (18), in which the force and position of the movable portion is controlled while elongation and vibration of the wires are prevented.

(19) The master-slave system according to Item (18),

in which the control apparatus constitutes a control system configured to independently control an acceleration response and tension of the wires in a control model in which the movable portion is driven by a pair of opposed motors with use of the wires, and controls the pair of motors, based on an acceleration reference value obtained from the control system.

(20) The master-slave system according to Item (19),

in which the control system has a center of gravity mode in which a motor C is controlled to make the movable portion achieve desired acceleration and a relative mode in which a motor R is controlled to make an elastic force that acts on the wires constant, performs acceleration control on the pair of motors, based on acceleration reference values for the motor C and the motor R.

(20-1) The master-slave system according to Item (20), in which kinematics of the motor C is expressed as center-of-gravity motion of the pair of motors, and kinematics of the motor R is expressed as relative motion of the pair of motors.

(21) The master-slave system according to Item (20), in which the control system controls, in the center of gravity mode, an estimated elastic force that acts from the motor C on the movable portion, to thereby prevent elongation and vibration of the wires.

(22) The master-slave system according to Item (21),

in which the control system includes a disturbance observer configured to estimate a disturbance on the movable portion, based on the estimated elastic force that acts from the motor C on the movable portion and a speed of the movable portion, in the center of gravity mode, and obtains the acceleration reference value for the motor C from the desired acceleration of the movable portion in consideration of the estimated elastic force and the disturbance estimated.

(23) The master-slave system according to Item (20), in which the control system controls, in the relative mode, the motor R so that predetermined tension acts on the wires.

(24) The master-slave system according to Item (24),

in which the control system includes a reaction force estimation observer configured to estimate an elastic force that acts on the wires by the motor R, in the relative mode, and obtains the acceleration reference value for the motor R, based on the predetermined tension and the elastic force estimated.

### REFERENCE SIGNS LIST

1: Master-slave system
10: Master apparatus
11: Input unit
12: Force presentation unit
20: Slave apparatus
21: Drive unit
22: State detection unit
30: Control system
100: Parallel wire apparatus
101 to 106: Wire
110: Movable portion
120: Rotatable portion
131 to 136: Linear actuator
141 to 148: Direction change pulley
500: One-degree-of-freedom model
502: Wire
503: First motor
504: Wire
505: Second motor
801: Actual motor acceleration control unit
802: Center of gravity mode control unit
803: Relative mode control unit
1001: To-be-controlled object
1002: Device acceleration control unit
1003: Estimated elastic force control unit
1101: To-be-controlled object
1102: Tension control unit
1103: Reaction force estimation observer (FROB)
1201: Acceleration controller
1202: Position controller

1. A control apparatus for a parallel wire apparatus configured to pull a movable portion with a plurality of wires,
the control apparatus being configured to control force and a position of the movable portion, based on acceleration.

2. The control apparatus according to claim 1,
wherein the control apparatus constitutes a control system configured to independently control an acceleration response and tension of the wires in a control model in which the movable portion is driven by a pair of opposed motors with use of the wires, and controls the pair of motors, based on an acceleration reference value obtained from the control system.

3. The control apparatus according to claim 2,
wherein the control system has a center of gravity mode in which a motor C is controlled to make the movable portion achieve desired acceleration and a relative mode in which a motor R is controlled to make an elastic force that acts on the wires constant, and performs acceleration control on the pair of motors, based on acceleration reference values for the motor C and the motor R.

4. The control apparatus according to claim 3, wherein the center of gravity mode includes a physical model of a two mass point system in which the motor C and the movable portion are connected to each other by a spring.

5. The control apparatus according to claim 3, wherein kinematics of the motor C is expressed as center-of-gravity motion of the pair of motors.

6. The control apparatus according to claim 3, wherein the relative mode includes a physical model in which the motor R pulls a spring with a resultant force of force generated by the pair of motors.

7. The control apparatus according to claim 3, wherein kinematics of the motor R is expressed as relative motion of the pair of motors.

8. The control apparatus according to claim 3, wherein the acceleration reference value for the motor C determined to make the movable portion achieve the desired acceleration and the acceleration reference value for the motor R determined to make the elastic force constant are subjected to coordinate transformation, to thereby obtain an acceleration reference value for the pair of motors.

9. The control apparatus according to claim 2, wherein the pair of motors each have mounted thereon a disturbance observer.

10. The control apparatus according to claim 2, wherein the pair of motors each have mounted thereon a reaction force estimation observer.

11. The control apparatus according to claim 3, wherein the control system controls, in the center of gravity mode, an estimated elastic force that acts from the motor C on the movable portion, to thereby prevent elongation and vibration of the wires.

12. The control apparatus according to claim 11,
wherein the control system includes a disturbance observer configured to estimate a disturbance on the movable portion, based on the estimated elastic force that acts from the motor C on the movable portion and a speed of the movable portion, in the center of gravity mode, and obtains the acceleration reference value for the motor C from the desired acceleration of the movable portion in consideration of the estimated elastic force and the disturbance estimated.

13. The control apparatus according to claim 3, wherein the control system controls, in the relative mode, the motor R so that predetermined tension acts on the wires.

14. The control apparatus according to claim 13,
wherein the control system includes a reaction force estimation observer configured to estimate an elastic force that acts on the wires by the motor R, in the relative mode, and obtains the acceleration reference value for the motor R, based on the predetermined tension and the elastic force estimated.

15. The control apparatus according to claim 3, wherein the control apparatus performs coordinate transformation on a mode space including the acceleration reference values for the motor C and the motor R in the control system, to thereby obtain an acceleration reference value for the pair of motors.

16. The control apparatus according to claim 15,
wherein positions of the pair of motors in a motor space are transformed to the mode space by coordinate transformation, and a position of the motor C calculated is fed back to the center of gravity mode of the control system, and
force generated by the pair of motors in the motor space is transformed to the mode space by coordinate transformation, and an estimated elastic force of the motor R calculated is fed back to the relative mode of the control system.

17. A control method for a parallel wire apparatus configured to pull a movable portion with a plurality of wires, the control method comprising steps of:
controlling, by a control system, a motor C in a center of gravity mode to make the movable portion achieve desired acceleration, the control system being configured to independently control an acceleration response and tension of the wires in a control model in which the movable portion is driven by a pair of opposed motors with use of the wires;
controlling, by the control system, a motor R in a relative mode to make an elastic force that acts on the wires constant; and
performing, by the control system, acceleration control on the pair of motors, based on acceleration reference values for the motor C and the motor R.

18. A master-slave system comprising:
a master apparatus and a slave apparatus at least one of which includes a parallel wire mechanism configured to pull a movable portion with a plurality of wires; and
a control apparatus configured to control force and a position of the movable portion, based on acceleration.

19. The master-slave system according to claim 18,
wherein the control apparatus constitutes a control system configured to independently control an acceleration response and tension of the wires in a control model in which the movable portion is driven by a pair of opposed motors with use of the wires, and controls the pair of motors, based on an acceleration reference value obtained from the control system.

20. The master-slave system according to claim 19,
wherein the control system has a center of gravity mode in which a motor C is controlled to make the movable portion achieve desired acceleration and a relative mode in which a motor R is controlled to make an elastic force that acts on the wires constant, and performs acceleration control on the pair of motors, based on acceleration reference values for the motor C and the motor R.
